(19) United States
(12) Patent Application Publication
(10) Pub. No.: US 2026/0096898 A1
(43) Pub. Date: Apr. 9, 2026

(54) CONVERTIBLE GLENOID IMPLANT

(71) Applicant: HOWMEDICA OSTEONICS CORP., Mahwah, NJ (US)

(72) Inventors: Brian C. Hodorek, Winona Lake, IN (US); Shawn Martin Gargac, Fort Wayne, IN (US)

(73) Assignee: HOWMEDICA OSTEONICS CORP., Mahwah, NJ (US)

(21) Appl. No.: 19/418,022

(22) Filed: Dec. 12, 2025

Related U.S. Application Data
(63) Continuation of application No. 16/894,149, filed on Jun. 5, 2020, now Pat. No. 12,527,667.
Application No. 16/894,149 is a continuation of application No. 15/058,045, filed on Mar. 1, 2016, now Pat. No. 10,722,374.
(60) Provisional application No. 62/157,059, filed on May 5, 2015.

(51) Int. Cl. A61F 2/40; A61F 2/30

(52) U.S. Cl. CPC A61F 2/4081; A61F 2002/30362; A61F 2002/30487; A61F 2002/30563; A61F 2002/30565; A61F 2002/30774; A61F 2002/30878; A61F 2002/4022; A61F 2002/4085; A61F 2220/0008

(57) ABSTRACT

A glenoid implant including a base plate and an articular component. The base plate can include a body and a support structure extending from a distal surface of the body. The body can include a plurality of openings. The articular component can be configured to removably couple to the base plate. The articular component can include a recessed portion configured to at least partially receive the body of the base plate. At least one engagement structure can protrude from a distal facing surface of the recessed portion. Each engagement structure can correspond to one of the plurality of openings in the body. A distal face of the articular component surrounding the recessed portion can be configured to abut the subchondral bone.

### INCORPORATION BY REFERENCE TO ANY PRIORITY APPLICATIONS

This application is a continuation of U.S. patent application Ser. No. 15/058,045, filed Mar. 1, 2016, which claims priority benefit under 35 U.S.C. § 119(e) to U.S. Provisional Application No. 62/157,059, filed May 5, 2015, which is hereby incorporated by reference in its entirety herein. Any and all applications for which a foreign or domestic priority claim is identified in the Application Data Sheet as filed with the present application are hereby incorporated by reference under 37 C.F.R. § 1.57.

### BACKGROUND

### Field

The present disclosure relates to a convertible glenoid implant of a shoulder joint prosthesis.

### Description of the Related Art

In a shoulder joint, the head of the humerus interacts with the glenoid cavity of the scapula in a manner similar to a “ball and socket” joint. Over time, it may become necessary to replace the shoulder joint with a prosthetic shoulder joint including a glenoid implant.

The glenoid implant typically includes an articular component that is adapted to articulate with the head of a humeral component, for example, the anatomical head of the humerus or a portion of a humeral implant. In some cases, the glenoid implant may provide an anatomical configuration in which the articular component includes a cavity that replaces the glenoid cavity. In other cases, the glenoid implant may provide a reversed configuration in which the articular component includes a hemispherical dome that cooperates with a complementary cavity defined by a humeral implant.

### SUMMARY

Some glenoid implants can include a base plate for fixing the articular body to the glenoid. The base plate can be permanently secured in the glenoid by the surgeon before an articular component is positioned on the base plate. In some cases, the glenoid implant needs to be replaced. The base plate remains coupled to the glenoid and the articular body is replaced. The surgeon may provide a new articular component of a different configuration. For example, the implant may be converted from an anatomical configuration to a reversed configuration.

When the base plate is positioned on the subchondral bone, the combination of the base plate and the articular component (e.g., a glenoid component or a reverse component) can be too thick and further displace the glenoid from the humeral component.

In some cases, a base plate can be at least partially loaded into the glenoid, such that a distal facing surface of the base plate is positioned in the glenoid and beyond the subchondral bone. However, milling the glenoid to insert the base plate can be difficult and require removal of a large portion of the bone. Further, loading the glenoid implant into the glenoid leaves no subchondral bone support behind the base plate. Since there is no subchondral support behind the glenoid implant, surgeons cannot lag screws to achieve the desired compression and the glenoid implant can subside. Thus, it can be desirable for the base plate to be positioned on the subchondral bone to avoid milling the bone. Positioning the base plate on the subchondral bone can also lag the screws into the glenoid to achieve initial fixation.

The base plate may include metal, such as a titanium alloy, while the articular body can include a synthetic material, such as polyethylene. However, the direct interaction between the metal base plate and synthetic articular body can cause the synthetic articular body to wear and displace the articular surface of the glenoid component. Thus, it may be desirable for a proximal surface of the base plate to be spaced apart from a distal facing surface of the articular component when the articular component is coupled to the base plate.

Certain aspects of the disclosure are directed toward a glenoid implant including a base plate and an articular component configured to removably couple to the base plate. The base plate can include a body and a support structure extending from a distal surface of the body. The body can include a plurality of openings. The articular component can include a recessed portion configured to at least partially receive the body of the base plate. At least one engagement structure can protrude from a distal facing surface of the recessed portion. Each engagement structure can correspond to one of the plurality of openings in the body. In certain aspects, a proximal surface of the base plate can be spaced apart from the distal facing surface of the recessed portion when the articular component is coupled to the base plate, and/or a distal face of the articular component surrounding the recessed portion can be configured to abut the subchondral bone.

Certain aspects of the disclosure are directed toward methods of implanting a glenoid implant. The method can include inserting a base plate into a glenoid cavity such that a support structure of the base plate is inserted into a subchondral bone portion and a body of the base plate is positioned thereon a subchondral bone surface. The body can include a plurality of openings. A screw can be advanced through one of the plurality of openings and into the subchondral bone. A first articular component can be secured to the base plate such that a recessed portion of the first articular component is advanced over the body of the base plate and an engagement member of the first articular component is inserted into a corresponding opening of the plurality of openings.

The above-mentioned method can further include removing the first articular component from the base plate and securing a second articular component to the base plate. The first articular component can be a glenoid component and the second articular component can be a reverse component.

Certain aspects of the disclosure are directed toward a glenoid system including a base plate, a glenoid component, and a reverse component. The base plate can include a body and a support structure extending from a distal surface of the body. The body can include a plurality of openings. The glenoid component can be configured to removably couple to the base plate and can include a recessed portion configured to at least partially receive the body of the base plate. A proximal surface of the base plate can be spaced apart from a distal facing surface of the anatomical recessed portion when the glenoid component is coupled to the base plate. The reverse component can be configured to removably couple to the base plate and can include a reverse recessed portion configured to at least partially receive the body of the base plate. The proximal surface of the base plate can be spaced apart from a distal facing surface of the reverse recessed portion when the reverse component is coupled to the base plate.

Any feature, structure, or step disclosed herein can be replaced with or combined with any other feature, structure, or step disclosed herein, or omitted. Further, for purposes of summarizing the disclosure, certain aspects, advantages, and features of the inventions have been described herein. It is to be understood that not necessarily any or all such advantages are achieved in accordance with any particular embodiment of the inventions disclosed herein. No individual aspects of this disclosure are essential or indispensable.

### BRIEF DESCRIPTION OF THE DRAWINGS

Various embodiments are depicted in the accompanying drawings for illustrative purposes, and should in no way be interpreted as limiting the scope of the embodiments. Furthermore, various features of different disclosed embodiments can be combined to form additional embodiments, which are part of this disclosure.

FIG. 1 represents a cross-sectional view of a prior art embodiment of modular glenoid implant.

FIG. 2A illustrates a bottom perspective view of an embodiment of a glenoid implant.

FIG. 2B illustrates a cross-section of the glenoid implant shown in FIG. 2A taken along line 2B-2B.

FIG. 2C illustrates an exploded view of the glenoid implant shown in FIG. 2A.

FIG. 2D illustrates an embodiment of a reverse component that can interface with the base plate shown in FIGS. 2A-2C.

FIG. 2E illustrates a cross-section of an embodiment of a glenoid implant including the reverse component shown in FIG. 2D.

FIG. 2F illustrates an exploded view of the reverse glenoid implant shown in FIG. 2E.

FIG. 3A illustrates a base plate secured to a glenoid.

FIG. 3B illustrates a glenoid component secured to the base plate shown in FIG. 3A. FIG. 3C illustrates a cross-section of the glenoid implant shown in FIG. 3B taken along line 3C-3C.

FIG. 3D illustrates a perspective view of the reverse glenoid implant of FIGS. 2D-2F secured to the glenoid.

FIG. 4A illustrates a cross-section of another embodiment of a glenoid implant.

FIG. 4B illustrates a bottom perspective view of the glenoid component shown in FIG. 3A.

FIG. 4C illustrates a bottom perspective view of the base plate shown in FIG. 3B.

FIG. 4D illustrates a bottom perspective view of an embodiment of a reverse component that can interface with the base plate shown in FIG. 4C.

FIG. 4E illustrates an exploded view of the glenoid implant shown in FIG. 4A.

FIG. 5A illustrates a cross-section of another embodiment of a glenoid implant.

FIG. 5B illustrates a bottom perspective view of the glenoid component shown in FIG. 4A.

FIG. 5C illustrates a bottom perspective view of the base plate shown in FIG. 4A.

FIG. 5D illustrates a bottom perspective view of another embodiment of a reverse component that can interface with the base plate shown in FIG. 4C.

FIG. 5E illustrates an exploded view of the glenoid implant shown in FIG. 5A.

FIG. 5F illustrates an exploded view of a reverse glenoid implant with the reverse component shown in FIG. 5D.

### DETAILED DESCRIPTION

Some glenoid implants can include a base component for fixing the articular body to the glenoid. The base component can be permanently secured in the glenoid by the surgeon, for example by using screws, before an articular component is positioned on the base plate. In some cases, the glenoid implant needs to be replaced. The base component remains coupled to the glenoid and the articular body is replaced. The surgeon may provide a new articular component of a different configuration. For example, the implant may be converted from an anatomical configuration to a reversed configuration.

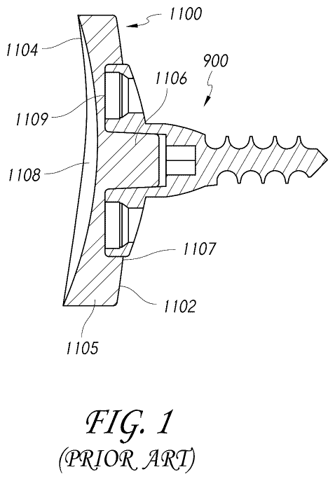

Glenoid components known in the art have typically been constructed from metal. FIG. 1 illustrates one such example of a glenoid component 1100 having a metal body 1105 and a metal base plate 900. The body 1105 defines circumferential recess 1107, which extends from the medial side 1102 towards the lateral side 1104. The tapered trunnion 1106 extends from the base 1109 of recess 1107 towards the medial side 1102, tapers from base 1009 to its proximal end, and is adapted to be received and attached to the tapered cavity 910 of the baseplate 900. Recess 1107 is adapted to receive a portion, or the entirety of, the body 902 of the baseplate 900.

### Single Engagement Structure

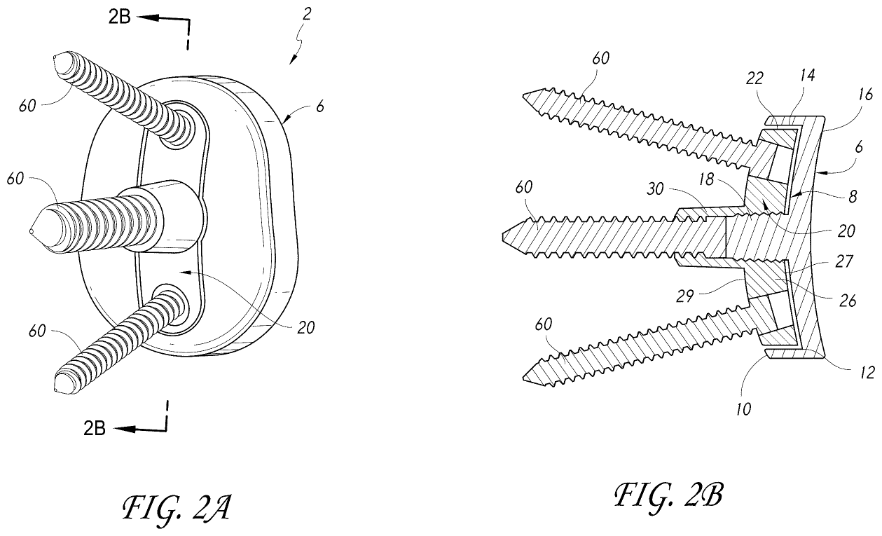

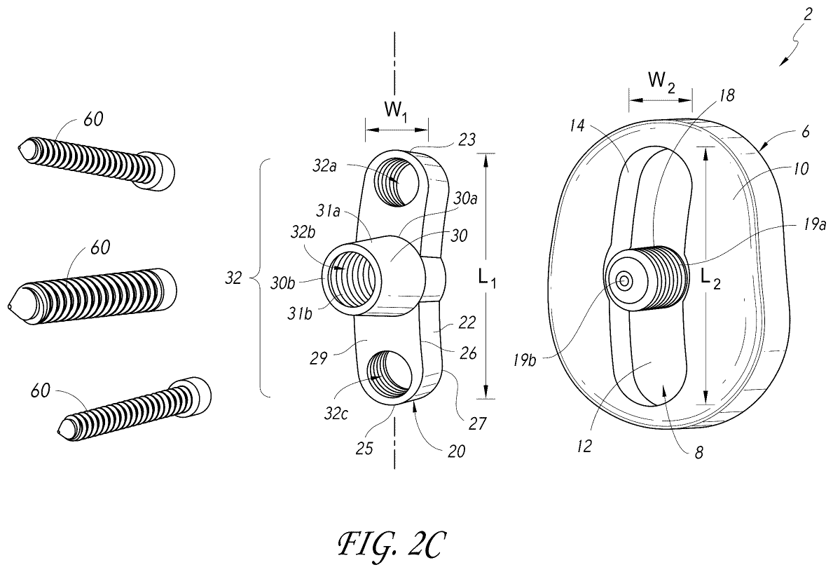

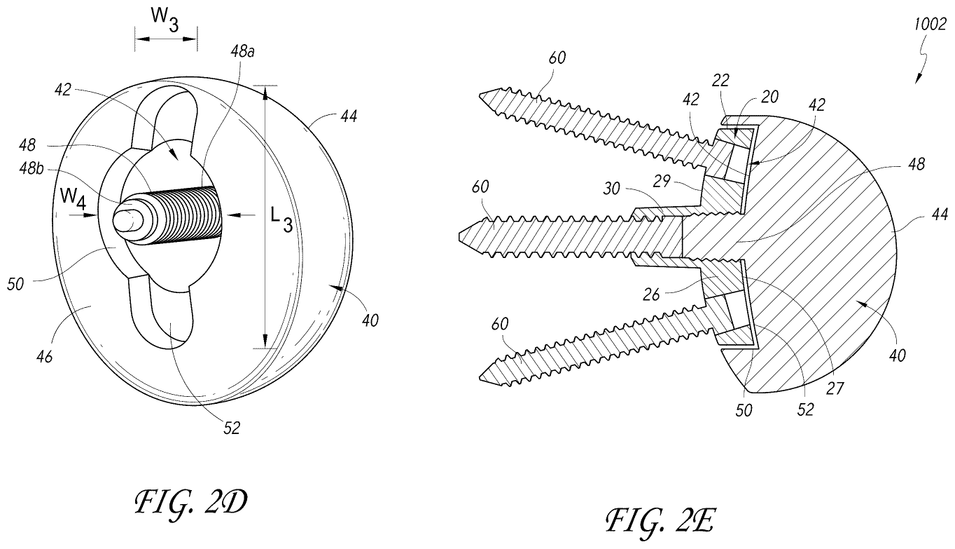

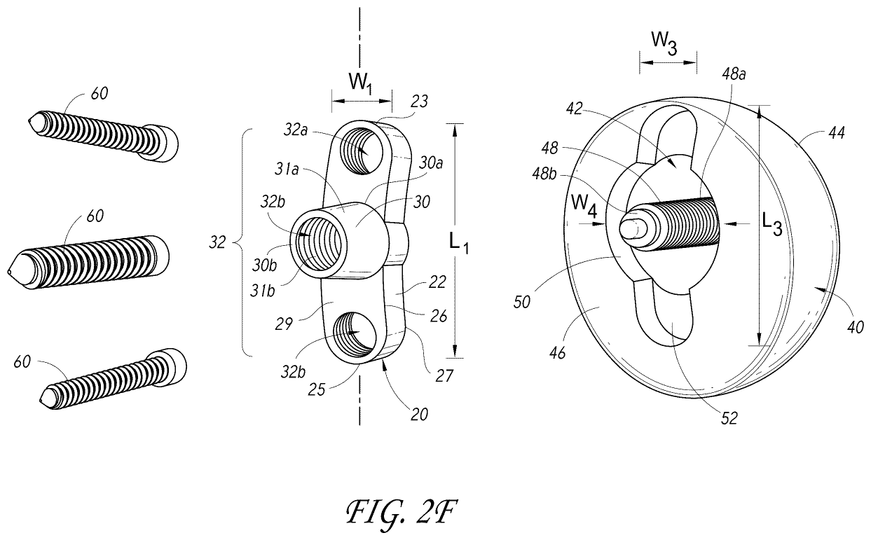

FIGS. 2A-2F illustrate glenoid implants 2, 1002 that can been implanted in a glenoid. Each of the glenoid implants 2, 1002 can include an articular component (e.g., a glenoid component 6 or a reverse component 40) removably secured to a base plate 20. For example, the glenoid implant 2 can include a glenoid component 6 configured to be removably coupled to the base plate 20 (see FIGS. 2A and 2B).

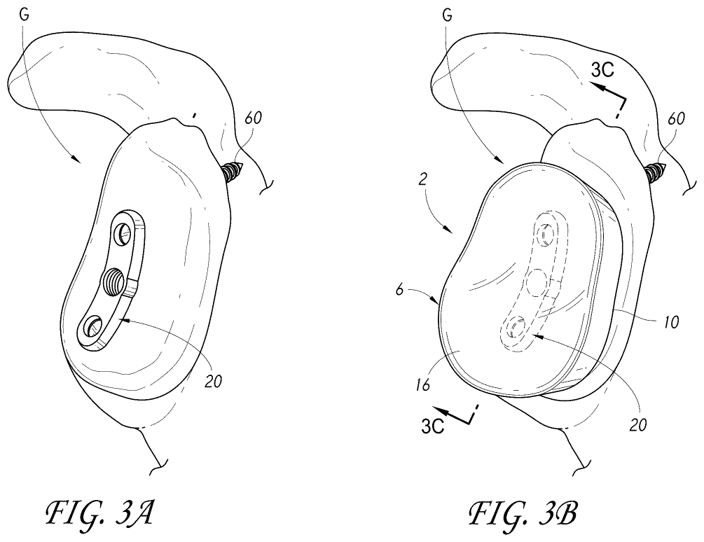

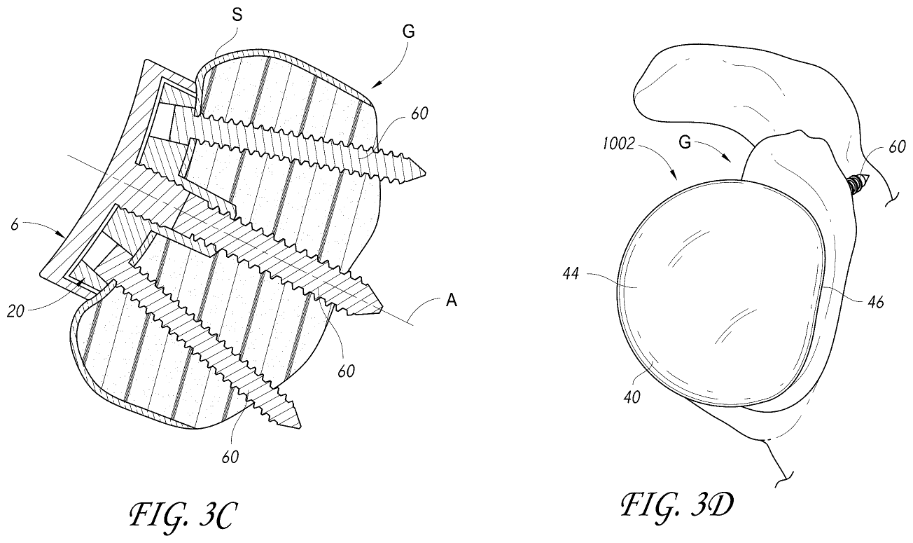

As shown in FIG. 2C, the base plate 20 can include a body 26 and a support structure 30. The body 26 can include an outer peripheral wall 22 having generally curved and/or flat surfaces. The outer peripheral wall 22 can extend between a lateral surface 27 (or proximal face) and a medial surface 29 (or distal face) of the body 26 (see FIG. 2C). A thickness of the body 26 can be defined from the lateral surface 27 to the medial surface 29 of the body 26. A length L₁ of the body 26 can be defined from a superior edge 23 of the body 26 to an inferior edge 25 of the body 26 (see FIG. 2C) and extend along an axis generally perpendicular to a longitudinal axis of the glenoid implant 2 when the implant 2 is secured to the glenoid (see FIGS. 3A and 3B). A width W₁ of the body 26 can be measured perpendicular to the length L₁.

The body 26 can be generally elongate. For example, the body 26 can be generally rectangular, generally elliptical, or any other elongate shape. A length L₁ of the body 26 can be substantially longer than a width W₁ of the body 26, e.g., the length L₁ can be at least about three times longer than the width W₁, at least about four times longer than the width W₁, or more (see FIG. 2C).

The body 26 can include a plurality of openings 32 (e.g., two, three, four, five, or more). At least some or all of the plurality of openings 32 can be aligned along the length L₁ of the body 26. For example, as shown in FIG. 2C, the body 26 can include three openings 32a, 32b, 32c aligned along the length L₁ of the body 26. One or more of the openings 32 may be threaded on an internal wall thereof and adapted to engage a screw 60 (e.g., having a single lead, double lead, triple lead, or other number of leads and/or right or left-handed threads).

The support structure 30 can extend from the medial surface 29 of the body 26 and extend generally perpendicular to the body 26 (see FIG. 2C). The support structure 30 can include a circumferential wall portion extending from a proximal portion 30a to a distal portion 30b of the support structure 30. The wall portion can include an exterior surface 31a and an interior surface 31b defining a lumen extending through the support structure 30. The exterior surface 31a can be generally smooth and without any threads, ribs, grooves, or other structures. The interior surface 31b can be threaded and adapted to engage a screw 60 and/or an engagement structure 18 of the glenoid component 6.

The lumen of the support structure 30 can be aligned with one of the openings 32. In some instances, as shown in FIG. 2C, the opening 32b aligned with the lumen of the support structure 30 can have a larger diameter than the peripheral openings 32a, 32c. Accordingly, a larger screw 60 can be advanced through the support structure opening 32b than the peripheral openings 32a, 32c.

As shown in FIG. 2C, the support structure 30 can taper from the proximal portion 30a toward the distal portion 30b. For example, the support structure 30 can be generally frustoconical and/or include a chamfer edge at the distal portion 30b of the support structure 30. In certain variants, the support structure 30 can be generally cylindrical and include a generally uniform diameter.

FIG. 2C also illustrates an embodiment of the glenoid component 6 adapted to engage the base plate 20. The glenoid component 6 can include a lateral surface 16 (or proximal face) and a medial surface 10 (or distal face). The medial surface 10 can have a recessed portion 8 with a peripheral wall 14 and a medial-facing surface 12 (or distal-facing surface). The recessed portion 8 can be shaped and sized to receive at least a partial thickness of the body 26. More particularly, when assembled, the lateral surface 27 of the base plate 8 can be positioned adjacent to and spaced away from the medial-facing surface 12 of the recessed portion 8.

The recessed portion 8 can have generally the same shape as the body 26 such that the recessed portion can at least partially or entirely receive the body 26 therein. For instance, the recessed portion 8 can have a generally elongate shape. A length L₂ of the recessed portion 8 can be substantially longer than a width W₂ of the body 26, e.g., the length L₂ can be at least about three times longer than the width W₂, at least about four times longer than the width W₂, or more. In embodiments, the shape of the recessed portion 8 is complementary to the body 26, such that the recessed portion is shaped to receive the body 26 therein. The recessed portion 8 can be centrally located with respect to the glenoid component 6.

The length L₂ and/or width W₂ of the recessed portion 8 can be greater than the length L₁ and/or width W₁ of the body 26, respectively (see FIG. 2C), such that a space remains between the peripheral walls 14 of the recessed portion 8 and the outer peripheral wall 22 of the body 26 when the glenoid implant 2 is fully assembled (see FIG. 2B). A thickness of the peripheral wall 14 of the recessed portion 8 can be greater than a thickness of the peripheral wall 22 the body 26, such that when the glenoid implant 2 is fully assembled, a space remains between the lateral surface 27 of the body 26 and the medial-facing surface 12 of the recessed portion 8. When the implant 2 is fully assembled, the medial surface 29 of the body 26 can be aligned or flush with the medial surface 10 of the glenoid component 6.

The spacing between the recessed portion 8 and the body 26 can prevent wear between the glenoid component 6 and the base plate 20, particularly when the base plate 20 comprises a metal (e.g., a titanium alloy) and when the glenoid component 6 comprises a polymer (e.g., polyethylene). Although not shown, the space between the recessed portion 8 and the body 26 can be filled with a shock absorbing material, such as polymers and copolymers, including, but not limited to silicones and polyurethanes.

At least one engagement structure 18 (e.g., one, two, three, four, five or more engagement structures) can extend from the medial-facing surface 12 of the recessed portion 8 and extend generally perpendicular to the length L₂ of the recessed portion 8. Each engagement structure 18 can be a peg, a barb, a screw, or other protruding structure, configured to be received by one of the openings 32 of the body 26 by a screw fit, snap fit, interference fit, or otherwise. As shown in FIG. 2C, the glenoid component 6 can include a single engagement structure 18 configured to interface with the opening 32b and/or the support structure 30.

Each engagement structure 18 can include an outer wall portion that can be threaded to threadably engage of the openings 32. The outer wall portion can extend from a proximal portion 19a to a distal portion 19b of the engagement structure 18. A length of the engagement structure 18 can be greater than a thickness of the recessed portion 8, such that the distal portion 19b of the engagement structure 18 extends distally of the medial surface 10 of the glenoid component 6. The distal portion 19b of the engagement structure 18 can be tapered to facilitate insertion of the engagement structure 18 into one of the openings 32 and/or the support structure 30.

As shown in FIGS. 2E and 2F, a reverse component 40 can interface with the base plate 20 shown in FIGS. 2A-2C to form the glenoid implant 1002. The reverse component 40 can include a medial surface 46 (or distal surface) and an articulating surface 44 configured to interface with a portion of a humeral component. The medial surface 46 can include a recessed portion 42 having a peripheral wall 50 and a medial-facing surface 52 (or distal-facing surface).

The recessed portion 42 can be eccentric with respect to the reverse component 40. For example, the recessed portion 42 can be closer to an inferior anatomic edge of the reverse component 40 than to a superior anatomic edge of the reverse component 40. The reverse component 40 can be placed more inferior on the glenoid surface than the glenoid component 6 to prevent scapular notching. The medial surface 46 of the reverse component 40 can be curved to fit the curve of the glenoid.

The recessed portion 42 can be shaped and sized to receive at least a partial thickness of the body 26. For instance, the recessed portion 42 can have a generally elongate shape. A L₃ of the recessed portion 42 can be substantially longer than a width W₃ of the body 26, e.g., the length L₃ can be at least about three times longer than the width W₃, at least about four times longer than the width W₃, or more. A width W₄ of a section of the recessed portion 42 surrounding the engagement structure 48 can be greater than the width W₃ at another section of the recessed portion 42. A thickness of the recessed portion 42 surrounding the engagement structure 48 can be greater than a thickness of the recessed portion 42 at other sections of the recessed portion 42.

The recessed portion 42 can be larger than the body 26, such a space remains between the recessed portion 8 and the body 26 when the implant 1002 is fully assembled (see FIG. 2E). For example, the lateral surface 27 of the body 26 can be spaced apart from the medial-facing surface 52 of the recessed portion 42 and/or the peripheral walls 22 of the body 26 can be spaced apart from the peripheral walls 50 of the recessed portion 42. When the implant 1002 is fully assembled, the medial surface 29 of the body 26 can be aligned or flush with the medial surface 46 of the reverse component 40 (See FIG. 2E). Although not shown, the space between the recessed portion 8 and the body 26 can be filled with a shock absorbing material, such as polymers and copolymers, including but not limited to silicone and polyurethane.

At least one engagement structure 48 (e.g., one, two, three, four, five or more engagement structures) can protrude from the medial-facing surface 52 of the recessed portion 42 and extend generally perpendicular to the length L₃ of the recessed portion 42. Each engagement structure 48 can be a peg, a barb, a screw, or other protruding structure, configured to be received by one of the openings 32 by a screw fit, snap fit, interference fit or otherwise. As shown in FIG. 2D, the reverse component 40 can include a single, engagement structure 48 configured to interface with the opening 32b and/or the support structure 30.

Each engagement structure 48 can include an outer wall portion that can be threaded to threadably engage of the openings 32. The outer wall portion can extend from a proximal portion 48a to a distal portion 48b of the engagement structure 48. A length of the engagement structure 48 can be greater than a thickness of the recessed portion 42, such that the distal portion 48b of the engagement structure 48 extends distally of the medial surface 46 of the reverse component 40. The distal portion 48b of each engagement structure 18 can be tapered to facilitate insertion of the engagement structure 18 into one of the openings 32 and/or the support structure 30.

FIGS. 3A-3C illustrate the glenoid implant 2 secured to the glenoid G. As shown in FIG. 3A, the base plate 20 can be secured to the glenoid G with the length of the body 26 extending in a superior-inferior direction. After the base plate 20 has been secured to the glenoid G using one or more screws 60, the glenoid component 6 can be secured over the base plate 20 (see FIG. 3B). When the glenoid component 6 is coupled to the base plate 20, a peripheral edge of the glenoid component 6 can form an entire peripheral edge of the glenoid implant 2. That is to say, the entire peripheral edge exterior to the glenoid G (e.g., from the lateral surface 16 to the medial surface 10 of the glenoid implant) can be the glenoid component 6 wherein the base plate 20 is captured between the glenoid component 6 and the glenoid G.

As shown in FIG. 2B, the medial surface 29 of the body 26 can be substantially flush with the medial surface 10 of the glenoid component 6 when the glenoid implant 2 is assembled. In this configuration, the medial surface 10 of the glenoid component 6 and the medial surface 29 of the body 26 can be configured to abut the subchondral bone S. That is to say, at least a portion of each of the medial surface 10 of the glenoid component 6 and the medial surface 29 of the body 26 are in direct contact with the subchondral bone S. When the medial surface 29 of the body 26 is positioned on the subchondral bone S, a surgeon can lag one or more screws 60 into the subchondral bone S to achieve the desired compression and fix the base plate 20 to the glenoid G. Additionally, since the medial surface 29 of the body can be positioned on the subchondral bone S, it is unnecessary to mill or ream the glenoid G before inserting the glenoid implant 2.

FIG. 3D illustrates a perspective view of the glenoid implant 1002 secured to the glenoid G for use in a reverse shoulder arthroplasty. The glenoid implant 1002 includes the reverse component 40, baseplate 20, and screws 60. The reverse component 40 can be secured to the same base plate 20 shown in FIGS. 3A-3C. When the reverse component 40 is secured to the base plate 20, the medial surface 46 of the reverse component 40 can be configured to abut the subchondral bone S.

### Multiple, Aligned Engagement Structures

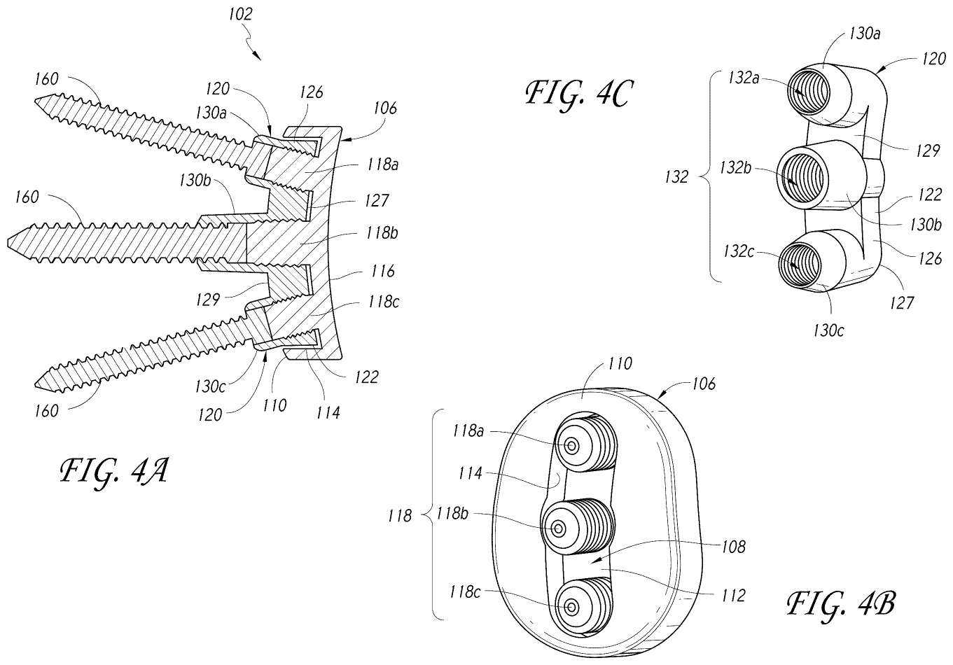

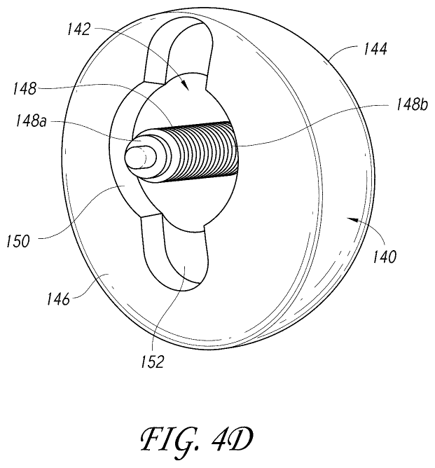

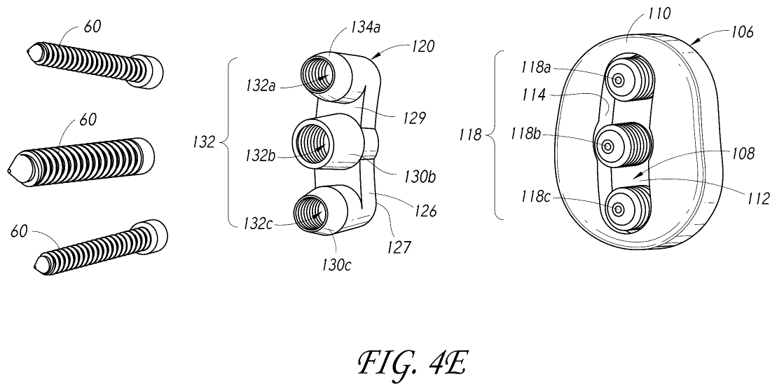

FIGS. 4A-4E illustrate additional embodiments of the glenoid components. The glenoid components resemble or are identical to the glenoid components discussed above except as described below. Accordingly, numerals used to identify features of the glenoid components shown in FIGS. 2A-2E are incremented by a factor of one hundred (100) to identify like features of the glenoid components shown in FIGS. 4A-4E. Any component or step disclosed in any embodiment in this specification can be used in other embodiments.

The glenoid component 106 can include a plurality of engagement structures 118 (e.g., two, three, four, five, or more engagement structures). Each engagement structure 118 can be similar to the engagement structure 18 described above. The number of the engagement structures 118 can be the same as the number of openings 132 in the base plate 120. At least some of the engagement structures 118 can be aligned along a transverse axis of the glenoid component 106.

As shown in FIGS. 4A and 4E, the glenoid component 106 can include three engagement structures 118a, 118b, 118c aligned along the length of the recessed portion 108. Each engagement structure 118a, 118b, 118c can interface with the respective corresponding opening 132a, 132b, 132c. The central engagement structure 118b can be longer than the peripheral engagement structures 118a, 118c.

The base plate 120 can include a plurality of support structures 130a, 130b, 130c extending from the medial surface 129 of the body 126. Each of the support structures 130a, 130b, 130c can include features similar to the support structure 30 described above and be aligned with one of the openings 132a, 132b, 132c. Each support structure 130a, 130b, 130c can be configured to receive a corresponding engagement structure 118a, 118b, 118c. A length of the support structures 130a, 130b, 130c can be sufficient to accommodate a proximal portion of a screw 160 and the corresponding engagement structure 118a, 118b, 118c (see FIG. 4A). A central support structure 130b can be longer than peripheral support structures 130a, 130c

FIG. 4D illustrates a reverse component 140 that can interface with the base plate 120 shown in FIG. 4C. As shown, the reverse component 140 can include a single engagement structure 148. However, in other embodiments, the reverse component 140 can include a plurality of engagement structures 148 (e.g., two, three, four, five, or more engagement structures). The number of engagement structures 148 can correspond to the number of openings 132 in the base plate 120. At least some of the engagement structures 148 can be aligned along a transverse axis of the reverse component 140. As one example, the reverse component 140 can include three engagement structure 148 aligned along the transverse axis of the reverse component 140.

### Lateral Engagement Structures

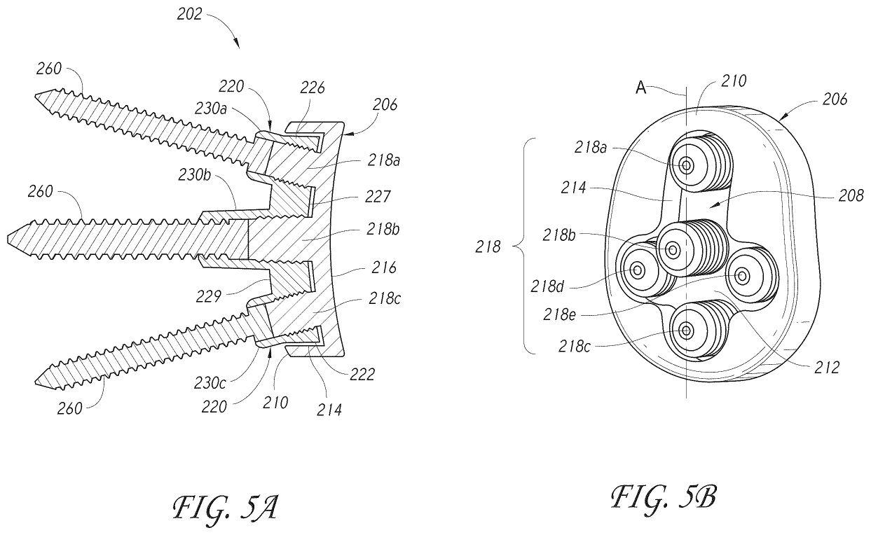

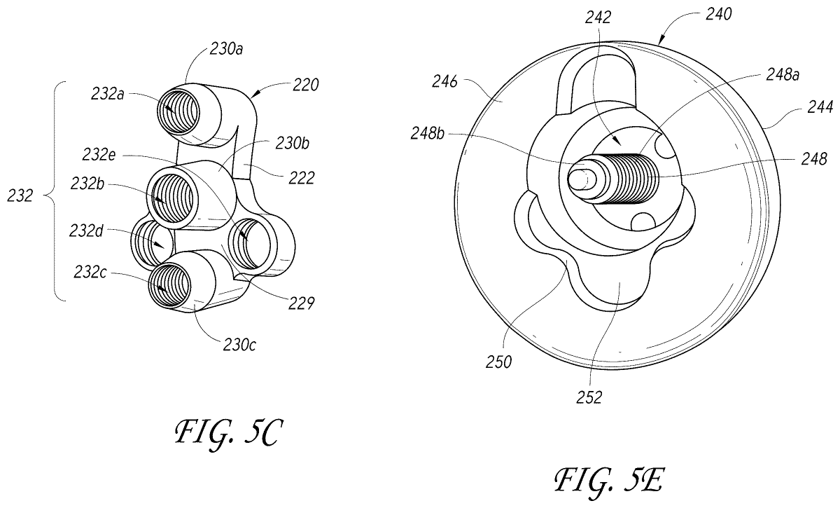

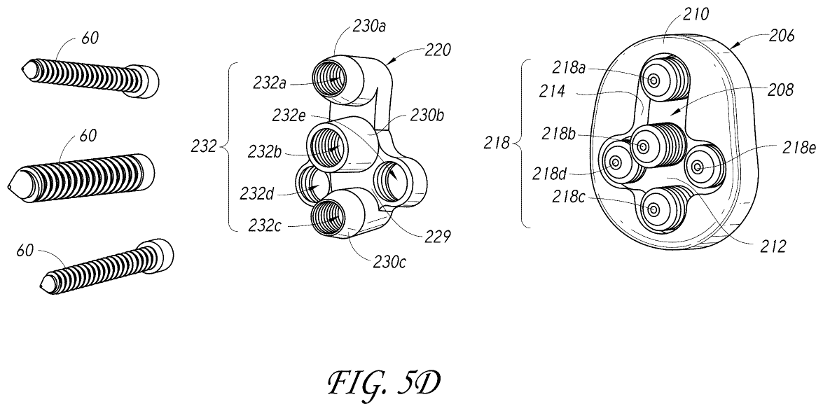

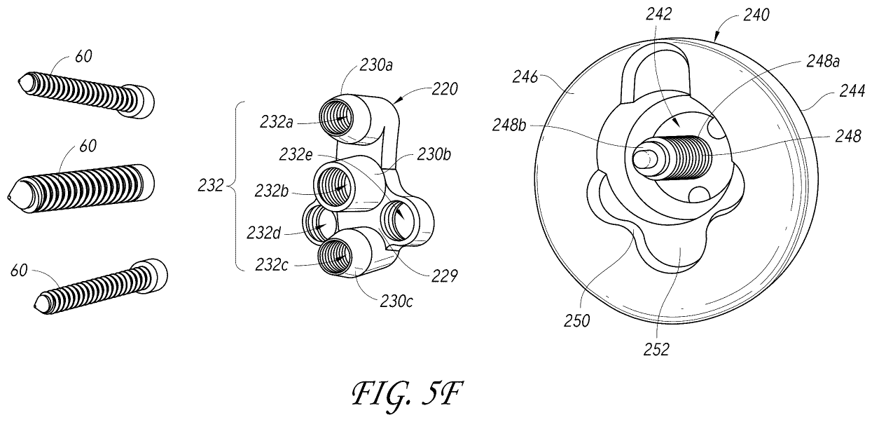

FIGS. 5A-5F illustrate additional embodiments of the glenoid components. The glenoid components resemble or are identical to the glenoid components discussed above in many respects. Accordingly, numerals used to identify features of the glenoid components shown in FIGS. 4A-4E are incremented by a factor of 100 hundred (100) to identify like features of the glenoid components shown in FIGS. 5A-5F. This numbering convention generally applies to the remainder of the figures. Any component or step disclosed in any embodiment in this specification can be used in other embodiments.

As shown in FIG. 5B, the glenoid component 206 can include a plurality of engagement structures 218 (e.g., two, three, four, five, or more engagement structures). Each engagement structure 218 can be similar to the engagement structure 18 described above. The number of engagement structures 218 can be the same as the number of openings 232 in the base plate 220 (see FIG. 5E). At least some of the engagement structures 218 can be aligned along a transverse axis of the glenoid component 206. For example, at least three engagement structures 218a, 218b, 218c can be aligned along the transverse axis of the glenoid component 206. Additional engagement structures 218d, 218e can be laterally displaced from the aligned engagement structures 218a, 218b, 218c and/or from a central transverse axis A (see FIG. 5B). The laterally displaced engagement structures 218d, 218e can be positioned such that the arrangement of engagement structures 218 is symmetrical about an axis extending through the aligned engagement structures 218a, 218b, 218c. The laterally displaced engagement structures 218d, 218e can be offset from the aligned engagement structures 218a, 218b 218c, for example, the laterally displaced engagement structures 218d, 218e can be positioned between the engagement structures 218b, 218c.

As shown in FIG. 5C, the base plate 220 can include laterally displaced openings 232d, 232e to accommodate the laterally displaced engagement structures 218d, 218e. For example, the base plate 220 can include three openings 232a, 232b, 232c aligned along the transverse axis of the base plate 220 and two openings 232d, 232e displaced from the aligned openings 232a, 232b, 232c. The laterally displaced openings 232d, 232e can be positioned such that the arrangement of openings 232 is symmetrical about the aligned openings 232a, 232b, 232c. The laterally displaced openings 232d, 232e can be offset from the aligned openings 232a, 232b, 232c, for example, the laterally displaced openings 232d, 232e can be positioned between the engagement structures 232b, 232c.

The base plate 220 can include a plurality of support structures 230a, 230b, 230c extending from the medial surface 229 of the body 226. Each of the support structures 230a, 230b, 230c can include features similar to the support structure 30 described above and can be aligned with one of the openings 232. At least some of the openings 232 can provide access to a corresponding support structure 230a, 230b, 230c. As shown in FIG. 5C, the support structures 230a, 230b, 230c can extend from the aligned openings 232a, 232b, 232c, but not the laterally displaced openings 232d, 232e. However, other quantities of support structures can be imagined, for example, the number of support structures can be the same as the number of openings.

FIG. 5E illustrates a reverse component 240 that can interface with the base plate 220 shown in FIG. 5C. The reverse component 240 can include a recessed portion 242 that can be sized to accommodate the base plate 230, including the laterally displaced openings 218e, 218d.

As shown in FIGS. 5E and 5F, the reverse component 240 can include a single engagement structure 248 similar to engagement structure 48. However, in other embodiments, the reverse component 240 can include a plurality of engagement structures 248 (e.g., two, three, four, five, or more engagement structures). The number of engagement structures 248 can correspond to the number of openings 232 and/or support structures 230a, 230b, 230c in the base plate 220. At least some of the engagement structures 248 can be aligned along a transverse axis of the reverse component 240, similar to the engagement structures 218a, 218b, 218c in FIG. 5B. As one example, the reverse component 240 can include three engagement structures 248 aligned along the transverse axis of the reverse component 240. As another example, a subset of the engagement structures 248 can be aligned along the transverse axis of the reverse component 240, while additional engagement structures 248 can be laterally displaced from the aligned engagement structures 248, e.g., to correspond with the arrangement of openings 232 in the base plate 220. Accordingly, the section of the recessed portion 248 accommodating the laterally displaced openings 232d, 232e can be wider than a remaining section of the recessed portion 248.

### Method of Implantation

Referring back to the glenoid implants 2, 1002 shown in FIGS. 3A-3D, in use, the base plate 20 can be secured to the glenoid G by inserting a support structure 30 of the base plate 20 into a subchondral bone portion S and positioning a medial surface 29 of the body 26 of the base plate 20 on the subchondral bone surface S. As shown in FIG. 3A, the body 26 can be positioned such that the length of the body 26 extends in the superior-inferior direction. A screw 60 can be advanced through at least one of the plurality of openings 32 of the body 26 and into the subchondral bone S.

The glenoid component 6 can be secured to the base plate 20 by advancing the recessed portion 8 of the glenoid component 6 over the body 26 of the base plate 20 and inserting an engagement member 18 of the glenoid component 6 into a corresponding opening of the plurality of openings 32 (e.g., the opening 32b extending through the support structure 30). A proximal portion of a screw 60 and the engagement structure 18 can be positioned in the support structure 30. A space is maintained between the lateral surface 27 of the body 26 and the medial-facing surface 12 of the recessed portion 8 and/or the peripheral wall 22 of the body 26 and the peripheral wall 14 of the recessed portion 8. After the glenoid component 6 is advanced over the base plate 30, the medial 10 of the glenoid component 6 can abut the subchondral bone S.

It may become necessary to remove the glenoid component 6 from the base plate, for example, if the glenoid implant 2 begins to wear or the bone further deteriorates. The reverse component 40 can be secured to the base plate 20 to form the glenoid implant 1002.

Although this method is described in the context of the glenoid components shown in FIGS. 3A-3D, a similar method can be used to implant glenoid implants 102 and 202.

### Terminology

Although certain embodiments have been described herein with respect to an anatomic component or a reverse component, the implants and methods described herein can interchangeably use any articular component, including the anatomic and reverse components described herein, as the context may dictate.

As used herein, the relative terms “proximal” and “distal” shall be defined from the perspective of the implant. Thus, proximal refers to the direction of the articular component and distal refers to the direction of the base plate when the implant is assembled.

Note that the terms “first” and “second”′ articular components can be used interchangeably and to refer to the anatomic components or the reverse components. Accordingly, the “first” and “second” openings can be used interchangeably and to refer to any one of the openings in the baseplate.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments.

The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

The terms “approximately,” “about,” and “substantially” as used herein represent an amount close to the stated amount that still performs a desired function or achieves a desired result. For example, the terms “approximately,” “about,” and “substantially” may refer to an amount that is within less than 10% of the stated amount, as the context may dictate. As an example, in certain embodiments, the term “generally perpendicular” refers to a value, amount, or characteristic that departs from exactly perpendicular by less than about 10 degrees.

Although certain embodiments and examples have been described herein, it will be understood by those skilled in the art that many aspects of the glenoid implants shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

Some embodiments have been described in connection with the accompanying drawings. However, it should be understood that the figures are not drawn to scale. Distances, angles, etc. are merely illustrative and do not necessarily bear an exact relationship to actual dimensions and layout of the devices illustrated. Components can be added, removed, and/or rearranged. Further, the disclosure herein of any particular feature, aspect, method, property, characteristic, quality, attribute, element, or the like in connection with various embodiments can be used in all other embodiments set forth herein. Additionally, it will be recognized that any methods described herein may be practiced using any device suitable for performing the recited steps.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

Any methods disclosed herein need not be performed in the order recited. The methods disclosed herein include certain actions taken by a practitioner; however, they can also include any third-party instruction of those actions, either expressly or by implication. For example, actions such as “inserting a base plate into a glenoid cavity” include “instructing insertion of a base plate into a glenoid cavity. ”

### EXAMPLE EMBODIMENTS

The following example embodiments identify some possible permutations of combinations of features disclosed herein, although other permutations of combinations of features are also possible.
1. A glenoid implant comprising:
   a base plate comprising a body and at least one support structure extending from a distal surface of the body, the body comprising a plurality of openings; and
   an articular component configured to removably couple to the base plate, the articular component comprising:
      a recessed portion configured to at least partially receive the body of the base plate; and
      at least one engagement structure protruding from a distal facing surface of the recessed portion, each of the at least one engagement structure corresponding to one of the plurality of openings in the body;
   wherein a proximal surface of the base plate is spaced apart from the distal facing surface of the recessed portion when the articular component is coupled to the base plate.
2. The implant of Embodiment 1, further comprising a shock absorbing material between the proximal surface of the base plate and the distal facing surface of the recessed portion.
3. The implant of Embodiment 2, wherein the shock absorbing material is selected from the group consisting of silicone and polyurethane.
4. The implant of any one of Embodiments 1 to 3, wherein the recessed portion is eccentric with respect to the articular component.
5. The implant of any one of Embodiments 1 to 4, wherein the recessed portion is closer to an inferior anatomic edge of the articular component than a superior anatomic edge of the articular component.
6. The implant of any one Embodiments 1 to 5, wherein a peripheral edge of the articular component forms an entire peripheral edge of the implant when the articular component is coupled to the base plate.
7. The implant of any one of Embodiments 1 to 6, wherein sidewalls of the recessed portion are spaced apart from side walls of the body when the articular component is coupled to the base plate.
8. The implant of any one of Embodiments 1 to 7, wherein the plurality of openings comprises a first opening, a second opening, and a third opening, and wherein the first opening, the second opening, and the third opening are axially aligned along a transverse axis of the body.
9. The implant of Embodiment 7, further comprising a fourth opening laterally displaced from the axially aligned openings.
10. The implant of any one of Embodiments 1 to 9, wherein the at least one support structure comprises a plurality of support structures, each of the plurality of openings of the body providing access to a corresponding support structure.
11. The implant of Embodiment 10, wherein each of the plurality of support structures corresponds to one of the at least one engagement structures.
12. The implant of any one of Embodiments 1 to 11, wherein the plurality of openings are configured for insertion of at least one screw into at least one of the plurality of openings.
13. The implant of any one of Embodiments 1 to 12, wherein the articular component is a reverse component or a glenoid component.
14. A glenoid implant comprising:
   a base plate comprising a body and a support structure extending from a distal surface of the body, a length of the body being greater than a width of the body, the body comprising a plurality of openings, the base plate comprising a metal; and
   an articular component comprising a polymer, the articular component configured to removably couple to the base plate, the articular component comprising:
      a recessed portion configured to at least partially receive the body of the base plate; and
      at least one engagement structure protruding from a distal facing surface of the recessed portion, each of the at least one engagement structure corresponding to one of the plurality of openings in the body,
      wherein a distal face of the articular component surrounding the recessed portion is configured to abut the subchondral bone.
15. The glenoid implant of Embodiment 14, wherein the distal surface of the body is configured to abut the subchondral bone.
16. The glenoid implant of Embodiment 14 or 15, wherein the base plate is configured such that at least one screw can be screwed through at least one of the plurality of openings and into the subchondral bone.
17. A method of implanting a glenoid implant in a glenoid cavity, the method comprising:
   inserting a base plate into a glenoid cavity such that a support structure of the base plate is inserted into a subchondral bone portion and a body of the base plate is positioned thereon a subchondral bone surface, the body comprising a plurality of openings;
   advancing a screw through one of the plurality of openings and into the subchondral bone; and
   securing a first articular component to the base plate such that a recessed portion of the first articular component is advanced over the body of the base plate and an engagement member of the first articular component is inserted into a corresponding opening of the plurality of openings.
18. The method of Embodiment 17, wherein securing the first articular component to the base plate comprises maintaining a space between a proximal surface of the body and a distal facing surface of the recessed portion.
19. The method of Embodiment 17 or 18, wherein securing the first articular component to the base plate comprises positioning a distal face of the first articular component to abut the subchondral bone.
20. The method of any one of Embodiments 17 to 19, wherein securing the first articular component comprises inserting the engagement member into said one of the plurality of openings housing the screw.
21. The method of any one of Embodiments 17 to 20, further comprising advancing each of a plurality of screws into each of the plurality of openings.
22. The method of any one of Embodiments 17 to 21, further comprising:
   removing the first articular component from the base plate; and
   securing a second articular component to the base plate,
   wherein the first articular component is a glenoid component and the second articular component is a reverse component.
23. A glenoid system comprising:
a base plate comprising a body and a support structure extending from a distal surface of the body, the body comprising a plurality of openings; and
an glenoid component configured to removably couple to the base plate, the glenoid component comprising an anatomical recessed portion configured to at least partially receive the body of the base plate, a proximal surface of the base plate being spaced apart from a distal facing surface of the anatomical recessed portion when the glenoid component is coupled to the base plate,
a reverse component configured to removably couple to the base plate, the reverse component comprising a reverse recessed portion configured to at least partially receive the body of the base plate, the proximal surface of the base plate being spaced apart from a distal facing surface of the reverse recessed portion when the reverse component is coupled to the base plate.

The following is claimed:

1. A system, comprising:
a base plate, including:
a first surface configured to face bone,
a second surface disposed on an opposite side of the base plate as the first surface,
a lateral surface extending between the first surface and the second surface,
a first support structure extending from the first surface, the first support structure defining a first lumen, and
a second support structure extending from the first surface, the second support structure defining a second lumen; and
a first articular component configured to be coupled to the base plate to form a first implant, the first articular implant including:
a first surface configured to face bone,
a second surface configured to face bone, the second surface of the first articular component being recessed within the first articular component relative to the first surface of the first articular component,
a first engagement structure extending from the second surface of the articular component, the first engagement structure sized and configured to be received within the first lumen defined by the first support structure of the base plate, and
a second engagement structure extending from the second surface of the articular component, the second engagement structure sized and configured to be received within the second lumen defined by the second support structure of the base plate.

2. The system of claim 1, further comprising:
a first screw having a first head and a first shaft, the first head of the first screw sized and configured to be disposed within the first lumen defined by the first support structure when the first engagement structure is disposed within the first lumen; and
a second screw having a second head and a second shaft, the second head of the second screw sized and configured to be disposed within the second lumen defined by the second support structure when the second engagement structure is disposed within the second lumen.

3. The system of claim 1, wherein the first articular component includes a convex articular surface.

4. The system of claim 1, wherein the first articular component includes a concave articular surface.

5. The system of claim 1, wherein the base plate is sized and configured to be received within a recess defined by the first articular component.

6. The system of claim 5, wherein, when the base plate is disposed within the recess defined by the first articular component, the second surface of the base plate is spaced apart from the second surface of the first articular component.

7. The system of claim 6, further comprising:
a shock absorbing material sized and configured to be disposed between the second surface of the base plate and the second surface of the first articular component when the base plate is disposed within the recess defined by the first articular component.

8. The system of claim 5, wherein a perimeter of the base plate has a shape that is complementary to a shape of a perimeter of the recess defined by the first articular component.

9. The system of claim 1, wherein the first surface of the base plate is configured to engage subchondral bone of a glenoid.

10. The system of claim 9, wherein the first surface of the first articular component is configured to engage subchondral bone and be flush with the first surface of the base plate when the first articular component is coupled to the base plate to form the first implant.

11. The system of claim 1, further comprising:
a second articular component configured to be coupled to the base plate to form a second implant.

12. The system of claim 1, wherein the base plate defines an opening sized and configured to receive a screw therein.

13. The system of claim 12, wherein the opening is displaced laterally with respect to the first support structure and the second support structure.

14. A glenoid implant, comprising:
an articular component, including:
a concave articular surface, and
a second surface disposed on an opposite side of the articular component from the concave articular surface,
a third surface disposed between the concave articular surface and the second surface of the articular component, and
a wall extending between the second surface of the articular component and the third surface of the articular component, the wall and the third surface of the articular component collectively defining a recess within the articular component, and
a first engagement structure extending from the third surface;
a base component sized and configured to be received within the recess defined by the articular component such that an entire periphery of the base component is disposed within and surrounded by the articular component, the base component including:
a first surface configured to engage a glenoid;
a second surface disposed on an opposite side of the base component as the first surface,
a lateral surface extending between the first surface and the second surface, and
a first support structure extending from the first surface of the base component, the first support structure defining a first lumen;
a first screw of a first type sized and configured to be received within the first lumen defined by the first support structure, and
a plurality of screws of a second type, each screw of the plurality of screws of the second type sized and configured to be received within a respective one of a plurality of openings defined by the base component,
wherein the first engagement surface is sized and configured to be disposed within the first lumen when the first screw of the first type is disposed within the first lumen.

15. The glenoid implant of claim 14, wherein each opening of the plurality of openings includes a thread configured to engage a thread of a respective one of the plurality of screws of the second type.

16. The glenoid implant of claim 14, wherein the first lumen defined by the first support structure and the plurality of openings are aligned linearly.

17. The glenoid implant of claim 14, wherein the articular component includes a plurality of engagement structures extending from the third surface of the articular component.

18. The glenoid implant of claim 17, wherein the first engagement structure has a length that is greater than a length of each engagement structure of the plurality of engagement structures.

19. The glenoid implant of claim 17, wherein the base component includes a plurality of support structures, each support structure of the plurality of support structures defining a lumen sized and configured to receive a respective one of the plurality of engagement structures extending from the third surface of the articular component.

20. The glenoid implant of claim 19, wherein:
the plurality of support structures and the first support structure are aligned linearly, and
the plurality of openings are offset laterally from the plurality of support structures.
